(19) United States
(12) Patent Application Publication
(10) Pub. No.: US 2013/0287697 A1
(43) Pub. Date: Oct. 31, 2013

(54) Pharmaceutical microsphere for embolization

(71) Applicant: NATIONAL CHENG KUNG UNIVERSITY, Tainan City (TW)

(72) Inventors: Xi-Zhang LIN, Tainan City (TW); Hong-Ming TSAI, Tainan City (TW); Yi-Sheng LIU, Tainan City (TW); Chueh-Kuan WANG, Tainan City (TW); Tzong-Shyng LEU, Tainan City (TW); Ping-Hen CHEN, Tainan City (TW); Li-Jhen WANG, Tainan City (TW); Po-Hsun TSENG, Tainan City (TW); Yu-Han LI, Tainan City (TW); Chiung-Yu CHEN, Tainan City (TW)

(73) Assignee: NATIONAL CHENG KUNG UNIVERSITY, Tainan City (TW)

(21) Appl. No.: 13/758,000

(22) Filed: Feb. 4, 2013

(30) Foreign Application Priority Data
Apr. 27, 2012 (TW) 101115152

(51) Int. Cl. A61L 24/04; A61L 24/00

(52) U.S. Cl. CPC A61L 24/04; A61L 24/00
USPC 424/9.4

(57) ABSTRACT

A pharmaceutical microparticle for embolization is disclosed, which includes: a thermoresponsive polymer, an enhancer, a contrast agent, and a solvent. The particle size of pharmaceutical microparticle for embolization is 100-750 μm. The pharmaceutical microparticle for embolization of the present invention is an effective drug carrier, and has biodegradable and X-ray imaging properties.

### BACKGROUND OF THE INVENTION

1. Field of the Invention

The present invention relates to a pharmaceutical microparticle for embolization, and particularly relates to a pharmaceutical microparticle for embolization suitable for use as an effective drug carrier, and has biodegradable and X-ray imaging properties.

2. Description of Related Art

Cancer has been the first leading cause on top 10 causes of death in Taiwan, wherein hepatoma is the first leading cause of death for men and the second leading cause of death for women. Therapeutic treatment of hepatoma includes transcatheter arterial embolization (TAE), percutaneous ethanol injection (PEI), cryotherapy, radiotherapy, and chemotherapy, and so on.

In the case of using transcatheter arterial embolization in hepatoma therapy, nutrition fed to the liver tumor tissue is almost entirely provided through the liver artery, therefore when the liver artery is blocked, normal liver tissue will be able to continue to survive and not become subject to necrosis because the normal liver tissue would still have portal veins to supply blood flow; in contrast, the liver cancer tissue would become necrotic due to nutrition deficiency.

Currently, the embolization compound for TAE includes a degradable material, such as gelatin; and non-degradable material, such as polyvinyl alcohol (PVA), vinyl based resin, drug eluting beads (DEB), and so on. Among these, gelatin is cheaper but cannot work to effectively carry chemotherapy drugs, thereby resulting in poor treatment effect. On the other hand, some non-degradable materials can carry chemotherapy drugs effectively but are expensive and cannot degrade in vivo, thereby resulting in new vascular formation to supply cancer cells and resulting to poor treatment effect. In addition, the above-mentioned embolization compounds do not possess X-ray imaging properties for tracking the position thereof.

Therefore, what is needed in the art is to develop a pharmaceutical microparticle for embolization having drug carrying ability as well as biodegradable and X-ray imaging properties, so as to improve success rate of TAE and reduce undesired side-effects for prolongation of patient's life.

### SUMMARY OF THE INVENTION

An object of the present invention is to provide a pharmaceutical microparticle for embolization having drug carrying ability as well as biodegradable and X-ray imaging properties, so as to improve success rate of TAE.

To achieve the above and other objects, the present invention provides a pharmaceutical microparticle for embolization, which includes: a thermoresponsive polymer, a first enhancer, a contrast agent, and a solvent, wherein the particle size of pharmaceutical microparticle for embolization is 100-750 μm, and preferably 150-350 μm.

The pharmaceutical microparticle for embolization according to the present invention may come in any shape, such as spherical shape, spherical-like shape, pyramidal shape, columnar shape, cubical shape, irregular shape, etc., and preferably spherical shape.

In addition, the pharmaceutical microparticle for embolization according to the present invention may further include a chemical drug. The chemical drug is not particularly limited and may be any pharmaceutical agent that can provide a therapeutic benefit to a subject, and preferably a radioactive element compound, a fat-soluble drug, or a water-soluble drug. Examples of drug for cancer therapy include doxorubicin, bevacizumab, sorafenib, irinotecan, thalidomide, resveratrol, curcumin, antibiotics, and so on.

Furthermore, the radioactive element compound may preferably be rhenium-188 radioactive element compound, yttrium-90 radioactive element compound, or holmium-166 radioactive element compound, but is not limited thereto, and any well-known compound having a therapeutic benefit to a subject may be used, for example, rhenium-118 radioactive element compound, strontium-89 radioactive element compound, iodine-125 element compound, and so on.

In the present invention, the radioactive element compound is not particularly limited, and may be rhenium-188-N,N′-1,2-ethanediylbis-L-cysteine diethylester (ECD), yttrium-90, or holmium-166, rhenium-188-1-hydroxy-1,1-ethylidene disodium phosphonate (HEDP), rhenium-188 radioactive liposomes, or iodine-125-5-iodo-2′-deoxyuridine (IUdR) etc., and preferably rhenium-188-N,N′-1,2-ethanediylbis-L-cysteine diethylester (ECD), yttrium-90, and holmium-166.

In the pharmaceutical microparticle for embolization according to the present invention, the type of the contrast agent is not particularly limited, and may be any known component serving as a contrast agent for as long as it can function as a contrast agent, and is preferably lipodol or BaSO₄.

In the pharmaceutical microparticle for embolization according to the present invention, the thermoresponsive polymer is present in an amount of 0.3-4.0 parts by weight, and the first enhancer is present in an amount present in an amount of 0.6-9.0 parts by weight; preferably, the thermoresponsive polymer is present in an amount present in an amount of 0.3-3.5 parts by weight, and the first enhancer is present in an amount present in an amount of 0.6-7.0 parts by weight; and more preferably, the more responsive polymer is present in an amount present in an amount of 0.3-0.4 parts by weight, and the first enhancer is present in an amount present in an amount of 0.6-0.7 parts by weight. Herein, the thermoresponsive polymer may be selected from the group consisting of polyethylene glycol (PEG), cetyl alcohol, glycerol monostearate, ethylene glycerol monostearate, poloxamer 188 (Pluronic F68), and myristyl alcohol; preferably, the thermoresponsive polymer may be a combination of glycerol monostearate, ethylene glycerol monostearate, and poloxamer 188 (Pluronic F68). In addition, the first enhancer is selected from the group consisting of stearic acid, polyethylene glycol (PEG), stearylamine, poly(lactic-co-glycolic acid) (PLGA), polyethylene oxide, α-cyclodextrin, and polycaprolactone. Preferably, the first enhancer is a combination including stearic acid and polycaprolactone.

In addition, the pharmaceutical microparticle for embolization according to the present invention may further comprise one selected from the group consisting of a thickener and a second enhancer. Preferably, the pharmaceutical microparticle for embolization may further comprise a thickener and a second enhancer. The thickener is present in an amount present in an amount of 0.00-0.05 parts by weight, preferably 0.05-0.1 parts by weight, and more preferably 0.05-0.06 parts by weight.

Herein, the thickener is at least one selected from the group consisting of lecithin, cholesterol, and dextrin; and preferably, cholesterol.

The second enhancer may be selected from the group consisting of stearic acid, polyethylene glycol (PEG), stearylamine, poly(lactic-co-glycolic acid) (PLGA), polyethylene oxide, α-cyclodextrin, and polycaprolactone.

The present invention also provides a pharmaceutical microparticle for embolization, comprising: a thermoresponsive polymer; an enhancer; a contrast agent; a solvent; a thickener, wherein the more responsive polymer is selected from the group consisting of glycerol monostearate, ethylene glycerol monostearate, polycaprolactone and polyether polyol; the enhancer is selected from the group consisting of polycaprolactone, stearic acid, and cetyl alcohol; the contrast agent and the solvent are lipodol; and the thickener is selected from the group consisting of cholesterol, lecithin, and dextrin.

In the pharmaceutical microparticle for embolization according to the present invention, the thermoresponsive polymer is present in an amount present in an amount of 0.3-3.5 parts by weight, the enhancer is present in an amount present in an amount of 0.6-7.0 parts by weight, the thickener is present in an amount present in an amount of 0.00-0.05 parts by weight. Preferably, the more responsive polymer is present in an amount of 0.3-0.4 parts by weight, the enhancer is present in an amount of 0.6-0.7 parts by weight, the thickener is present in an amount of 0.00-0.05 parts by weight.

Accordingly, the components included in the pharmaceutical microparticle for embolization according to the present invention are common components used for current clinical pharmaceuticals. Therefore, comparing with other newly developed pharmaceuticals, the pharmaceutical microparticle for embolization according to the present invention may shorten the clinical trial period and accelerate application in clinical medicine.

The pharmaceutical microparticle for embolization according to the present invention may be prepared by any known process, and preferably spray granulation. Since spray granulation has the advantages of instant drying, high product quality, multi-level drying, and simple process etc., such a process has been widely used in pharmaceutical, chemical, material, food, and cosmetic industry.

The pharmaceutical microparticle for embolization according to the present invention has effective drug carrying ability, and biodegradable and X-ray imaging properties. The X-ray imaging property may be used to observe the stationary position of the pharmaceutical microparticle for embolization, and after injection of the pharmaceutical microparticle for embolization into the subject, an image can be taken directly by a X-ray imager, thereby confirming the arrival of the drug at the target site. In addition, the biodegradable property may inhibit the long-term accumulation of embolism in the extracellular matrix, and the blood vessel will be embolized by the microparticles after the injection of the pharmaceutical microparticle for embolization into the subject causing death of the cancer cell due to nutrient deficiency. After a period of time, the microparticles will degrade, and the remaining cancer cells still use the same blood vessel. Therefore, it may avoid the cancer cell from angiogenesis or transferring to other sites in the body by using another blood vessel. Furthermore, the pharmaceutical microparticle for embolization according to the present invention may carry a chemical drug to the target sited and slowly release the drug to improve the treatment and ease the patient's conditions efficiently.

Thus, the pharmaceutical microparticle for embolization according to the present invention may improve success rate of TAE, reduce undesired side effects for prolongation of patient's life, and can be used to treat liver cancer, kidney cancer, uterine fibroids, spleen embolism, and so on in clinic.

### BRIEF DESCRIPTION OF THE DRAWINGS

FIG. 1 shows the granulation system of the synthetic example according to the present invention.

FIG. 2A shows a cross-sectional diagram of the atomizing nozzle used in the monodispersed particle generation according to the present invention.

FIG. 2B shows a schematic diagram of the porous structure of the atomizing nozzle used in the monodispersed particle generation according to the present invention.

FIG. 3 shows a schematic diagram of the atomizing nozzle of the binary-fluid spray granulation according to the present invention.

FIG. 4 shows the photomicrograph of the microparticle produced by the monodispersed particle generation according to the present invention.

FIGS. 5A and 5B show the photomicrograph of the microparticle produced by the binary-fluid type atomizing process according to the present invention.

### DETAILED DESCRIPTION OF THE PREFERRED EMBODIMENT

Hereinafter, exemplary embodiments of the present invention will be described in detail. However, the present invention is not limited to the embodiments disclosed below, but can be implemented in various forms. The following embodiments are described in order to enable those of ordinary skill in the art to embody and practice the present invention, and those skilled in the art will appreciate that various modifications, additions and substitutions are possible without departing from the scope and spirit of the invention as disclosed in the accompanying claims.

### SYNTHETIC EXAMPLE

### Preparation of Pharmaceutical Microparticle for Embolization

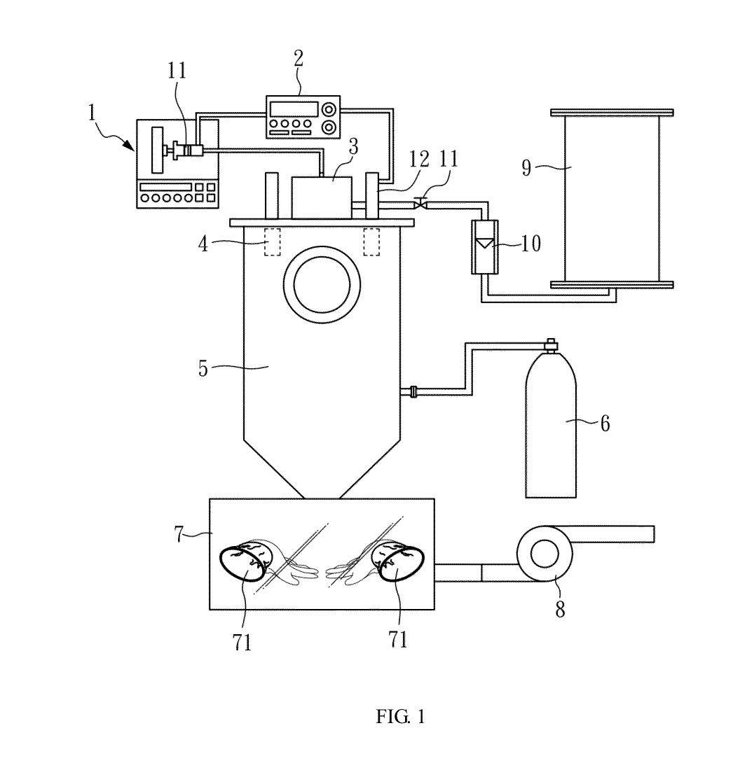

FIG. 1 shows a granulation system used in the synthetic example. As shown in FIG. 1, the experimental equipment included: a pharmaceutical agent feeding system driven by an injection pump 1 to control the feeding rate during the propulsion process; a heating system using a soft electric heating sheet to heat the thermoresponsive pharmaceutical agent and using a hot water bath to preserve heat and ensure a predetermined temperature during feeding, making sure that the feeding material is liquid and flowable; an atomizing nozzle system 3 which may be an external excitation porous system or a binary-fluid atomizing system; a sterilization device using a UV germicidal lamp 4 to continuously irradiate from the roof of spray granulation chamber to keep the chamber and the materials for embolization sterile; spray drying chamber 5 employing liquid nitrogen to produce dry cooling gas through an evaporator 6, wherein a cold blast was supplied from the side edge of the spray drying chamber 5 through a HEPA gas filter, to ensure that the entered cooling gas was sterile and clean, and the thermosensitive microparticle for embolization will form into a spherical cured particle during the flight path in cool air after sprayed by the atomizing nozzle system. The spray drying chamber 5 was made of stainless steel and surface-treated by electrolysis to maintain the requirements for pharmaceutical equipment. The experimental equipment also included: a collecting and packaging device 7 having a collection bucket as an atmosphere control system and a collection sheath 71 to avoid the embolic product from contamination during the collecting and packaging process; and an exhaust system using an exhaust fan 8 to discharge the gas in the chamber and filter out the microparticle for embolization, and the discharged gas was collected and processed according to regulations for medical waste disposal.

First, the components of microparticles were thoroughly mixed uniformly in amounts as listed in Table 1.

TABLE 1

| component | g/ml | function |
|---|---|---|
| lipodol | 0.5 ml-1.0 ml | Contrast agent & |
| | | solvent |
| Cetyl alcohol | 0.15 g-0.20 g | Thermo responsive |
| | | polymer |
| Glycerol monostearate | 0.20 g-0.30 g | Thermo responsive |
| | | polymer |
| Polyethylene glycol (PEG) | 0.20 g-0.25 g | Thermo responsive |
| | | polymer |
| Ethylene glycerol monostearate | 0.20 g-0.25 g | Thermo responsive |
| | | polymer |
| Stearic acid | 0.35 g-0.40 g | Enhancer |
| Polycaprolactone | 0.25 g-0.30 g | Enhancer |
| Cholesterol | 0.05 g-0.10 g | thickener |
| Dextrin | 0.00 g-0.05 g | thickener |
| Total weight | 1.50 g | |

Then, the syringe 11 and atomizing nozzle 3 of the injection pump 1 were heated to a temperature of 60-75° C., and maintained in such a temperature range.

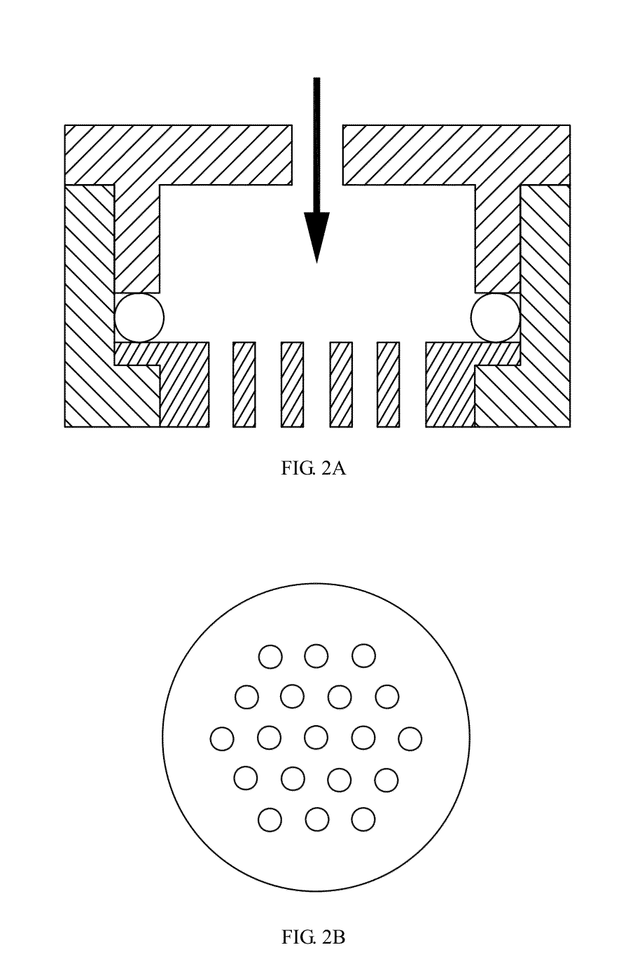

After that, the mixed microparticle raw materials were injected into the granulation apparatus in a feeding rate of 10 ml/min and melted into liquid form by a hot water bath, and then in monodispersed particle generation, the liquid raw materials were directly injected into the pressure atomizing nozzle 3 under application of external excitation to form the microparticles having a uniform particle diameter. The atomizing nozzle used in the monodispersed particle generation was shown in FIGS. 2A and 2B. Referring to FIGS. 2A and 2B, FIG. 2A shows a cross-sectional diagram of the atomizing nozzle used in the monodispersed particle generation; FIG. 2B shows a schematic diagram of the porous structure of the atomizing nozzle used in the monodispersed particle generation. The pharmaceutical agent feeding system is as shown in FIG. 1. In addition, an external acoustic excitation was applied to obtain the pharmaceutical microparticles for embolization having a uniform particle diameter. The direction of the arrowhead in FIG. 2A represents the feeding direction.

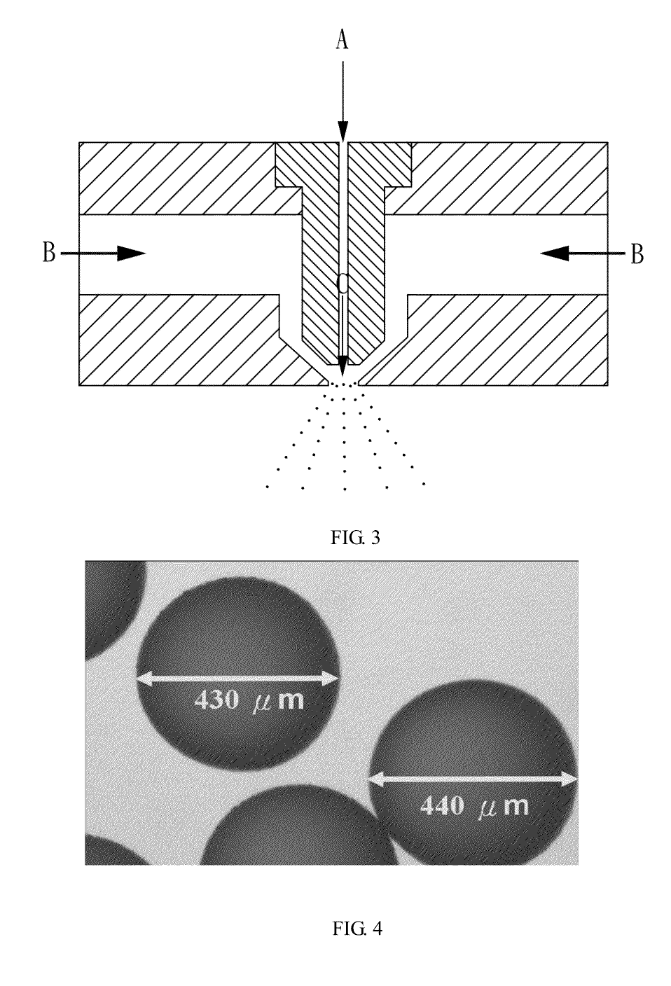

Further, in the binary-fluid type process, the spray was conducted through the atomizing nozzle 3 at a gas input rate of 30 L/min to obtain the embolization-oriented pharmaceutical microparticles. The atomizing nozzle of the binary-fluid spray granulation is shown in FIG. 3. FIG. 3 is a schematic diagram of the atomizing nozzle of the binary-fluid spray granulation, wherein the pharmaceutical agent feeding system and the heating system are the same as in the spray granulation technique, while the energy for spray vibration is provided by the gas from both sides to create finer microparticles for embolization. In FIG. 3, the direction of the arrowhead A represents the feeding direction, the direction of the arrowhead B represents the direction of gas input, and the direction of the arrowhead C represents the spray direction.

In this example, the mixture may pass through the UV germicidal lamp 4 and the gas filter to produce sterile microparticles.

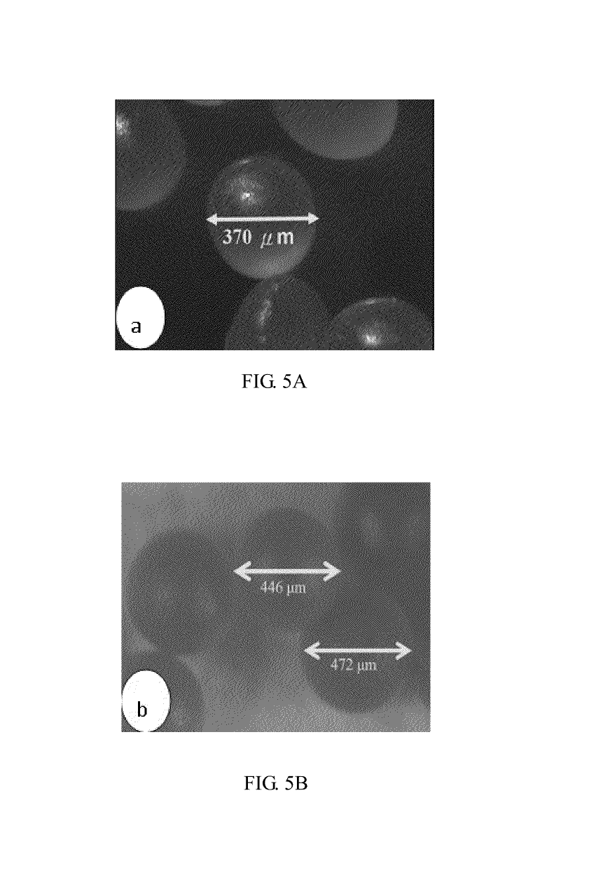

Finally, the product of the pharmaceutical microparticles for embolization was collected by the collecting and packaging device 7, dried by the exhausters 8, and then pictured by a microscope. The particle size of the microparticle in the picture was measured according to the scale bar. The photomicrograph of the microparticle produced by the monodispersed particle generation is shown in FIG. 4. The photomicrograph of the microparticle produced by the binary-fluid type atomizing process is shown in FIGS. 5A and 5B.

TABLE 2

| | | | | Atomizing | Particle |
|---|---|---|---|---|---|
| | Feeding | Gas input | syringe | nozzle | size of the |
| | rate | rate | temperature | temperature | product |
| Monodispersed | 1.0 ml/min-10 ml/min | 0 ml/min-20 ml/min | 75° C. | 75° C. | 400-500 μm |
| particle | | | | | |
| generation | | | | | |
| Binary-fluid | 10 | 30 L/min | 75 | 75° C. | 50-150 μm |
| type process | | | | | |

As shown in the results of the synthetic examples 1-3, the preparation method can produce the sterile microparticles having a uniform particle size without aggregation. In addition, such microparticles have a low degradation rate, and the effect of slow drug release can be realized when encapsulating a chemical drug. Furthermore, this preparation method has a high yield and without pollution from organic solvents. In addition, our microspheres have drug delivery ability, as well as biodegradable and X-ray imaging properties, which are useful in clinical practice.

The making and using of the embodiments of the disclosure are discussed in detail below. It should be appreciated, however, that the embodiments provide many applicable inventive concepts that can be embodied in a wide variety of specific contexts. The specific embodiments discussed are merely illustrative, and do not limit the scope of the disclosure.

What is claimed is:

1. A pharmaceutical microparticle for embolization, comprising:
a thermoresponsive polymer;
a first enhancer;
and a contrast agent;
wherein the pharmaceutical microparticle for embolization has a particle size of 100-750 μm.

2. The pharmaceutical microparticle for embolization of claim 1, having a particle size of 200-400 μm.

3. The pharmaceutical microparticle for embolization of claim 1, further comprising a chemical drug.

4. The pharmaceutical microparticle for embolization of claim 1, wherein the chemical drug is a radioactive element compound, a fat-soluble drug, or a water-soluble drug.

5. The pharmaceutical microparticle for embolization of claim 4, wherein the radioactive element compound is rhenium-188 radioactive element compound, yttrium-90 radioactive element compound, or holmium-166 radioactive element compound.

6. The pharmaceutical microparticle for embolization of claim 5, wherein the radioactive element compound is rhenium-188-N,N′-1,2-ethanediylbis-L-cysteine diethylester (ECD), yttrium-90, or holmium-166.

7. The pharmaceutical microparticle for embolization of claim 1, wherein the contrast agent is lipodol or BaSO₄.

8. The pharmaceutical microparticle for embolization of claim 1, wherein the thermo responsive polymer is present in an amount of 0.3-0.4 parts by weight, and the first enhancer is present in an amount of 0.6-0.7 parts by weight.

9. The pharmaceutical microparticle for embolization of claim 1, wherein the thermo responsive material is selected from the group consisting of polyethylene glycol (PEG), cetyl alcohol, glycerol monostearate, ethylene glycerol monostearate, poloxamer 188 (Pluronic F68), polycaprolactone, and myristyl alcohol.

10. The pharmaceutical microparticle for embolization of claim 1, wherein the first enhancer is selected from the group consisting of stearic acid, polycaprolactone, polyethylene glycol (PEG), cetyl alcohol, stearylamine, poly(lactic-co-glycolic acid) (PLGA), polyethylene oxide, and α-cyclodextrin.

11. The pharmaceutical microparticle for embolization of claim 1, further comprising at least one selected from the group consisting of a second enhancer and a thickener.

12. The pharmaceutical microparticle for embolization of claim 11, wherein the thickener is present in an amount of 0.00-0.05 parts by weight.

13. The pharmaceutical microparticle for embolization of claim 1, wherein the thickener is at least one selected from the group consisting of lecithin, cholesterol, and dextrin.

14. The pharmaceutical microparticle for embolization of claim 11, wherein the second enhancer is selected from the group consisting of stearic acid, polyethylene glycol (PEG), stearylamine, poly(lactic-co-glycolic acid) (PLGA), polyethylene oxide, α-cyclodextrin, and polycaprolactone.

15. A pharmaceutical microparticle for embolization, comprising:
a thermo responsive polymer;
an enhancer;
a contrast agent; and
a thickener
wherein the thermo responsive polymer is selected from the group consisting of glycerol monostearate, ethylene glycerol monostearate, polycaprolactone and polyether polyol; the enhancer is selected from the group consisting of polycaprolactone, stearic acid, and cetyl alcohol; the contrast agent and the solvent are lipodol; and the thickener is selected from the group consisting of cholesterol, lecithin, and dextrin.

16. The pharmaceutical microparticle for embolization of claim 15, wherein the thermo responsive polymer is present in an amount of 0.3-0.4 parts by weight, the enhancer is present in an amount of 0.6-0.7 parts by weight, the thickener is present in an amount of 0.00-0.05 parts by weight.
